SEQUENCE LISTING (7 sequences)

SEQ ID NO: 1 - DNA, 2343 bp, Arabidopsis thaliana
atggtggaagaagatttaaaccagattggtggtaatagtaattacaatggagaaggaggcgacccagagagcaacacacttaaccaacctctagttaaggctaatcgaacactttcttcaactccacttgctttggttggtgccaaagtttcccatatcgaaagcttggactatgaaataaacgagaacgatctgtttaagcatgattggagaaaaagatcaaaggcacaagtacttcaatacgtgttcttgaaatggacgttagcttgtcttgttggtcttttcactggtttaatcgctactctcatcaacttagctgttgaaaacatcgccggctataagcttttagccgttggtcacttcctcactcaagaaagatatgttacaggtctgatggtgcttgttggggcgaatttgggactgacgttggtggcgtctgtgctttgtgtgtgctttgctcctacggcggctggacctggaatccctgagatcaaagcttatcttaatggtgtagatactcccaacatgtttggtgctactactatgatcgttaagattgttggaagcattggagcggttgcagctggacttgatctaggtaaagagggtcctctagttcacattggaagctgcatagcttctttgcttggacaaggtggaacagacaaccaccgtatcaagtggcggtggcttcgttacttcaacaacgatagagaccgcagggatctgattacatgtggctcagctgcaggagtgtgtgcagccttcaggtcacctgttggaggtgtacttttcgccctcgaggaagttgctacttggtggagaagtgccttattgtggcggactttcttcagcacagcggttgttgtggttgttctaagagagttcatagagatctgcaattcagggaagtgtgggttgtttggaaaaggagggctaatcatgtttgatgtgagtcatgtaacttatacttaccatgtaactgatataatccctgtcatgttgattggtgtaatcggtggaattcttgggagcctgtacaatcatcttctgcataaagttctcaggctttacaatctcatcaatgagaagggtaagatccataaggtgcttctcagtcttacagtatcactctttacatctgtttgcctttatggccttcctttcttagcgaaatgcaagccttgtgacccctcgatagatgagatatgcccgacgaatggaagatcgggtaacttcaaacagttccattgccctaaaggttactacaatgatctagctactctgcttctcaccaccaacgatgatgctgtcagaaaccttttctcttccaacactcccaatgagtttggtatgggttccctttggatattctttgtgctatactgcatcttggggcttttcacatttggtattgcaacaccgtctggtctcttcctccccatcatcctcatgggtgctgcatatggccgaatgcttggcgctgcaatgggatcatacacaagtattgaccaagggctttatgctgtccttggtgcagctgcactcatggctggatcgatgagaatgactgtgtcactctgtgttatattccttgaactcaccaacaaccttcttttgcttcctataacgatgatcgtgcttctgatagccaaaactgtgggagacagctttaacccgagtatatatgacatcatcttgcatctaaagggcttacctttcttagaagcaaatccagagccgtggatgaggaacctcaccgttggtgagcttggtgatgctaagcccccggttgtaaccctgcaaggtgttgaaaaggtttcaaatatagttgatgtgctaaagaacacgacgcataatgcattccctgttttagatgaagcagaagtacctcaagtgggtctagcaactggggctacagaactccacgggttgatcttgagagcgcacctcgttaaagttctgaaaaagagatggttcttgacagagaaaagaagaacagaggagtgggaggtcagagaaaagtttccatgggatgaattggctgaaagagaagacaactttgacgacgtggccatcacaagcgctgaaatggaaatgtatgtcgatcttcatcctctcaccaacacaacaccttacacagtcatggagaacatgtcagtggccaaggctttagtacttttccggcaagtgggactccggcatttgcttattgttcccaagattcaagcctcaggaatgtgtcctgtggtagggatcttaaccagacaggacctaagggcatacaacattctacaagcctttcctctcttggaaaaatccaaaggtggaaagacacattga

SEQ ID NO: 2 - protein, 780 aa, Arabidopsis thaliana
MVEEDLNQIGGNSNYNGEGGDPESNTLNQPLVKANRTLSSTPLALVGAKVSHIESLDYEINENDLFKHDWRKRSKAQVLQYVFLKWTLACLVGLFTGLIATLINLAVENIAGYKLLAVGHFLTQERYVTGLMVLVGANLGLTLVASVLCVCFAPTAAGPGIPEIKAYLNGVDTPNMFGATTMIVKIVGSIGAVAAGLDLGKEGPLVHIGSCIASLLGQGGTDNHRIKWRWLRYFNNDRDRRDLITCGSAAGVCAAFRSPVGGVLFALEEVATWWRSALLWRTFFSTAVVVVVLREFIEICNSGKCGLFGKGGLIMFDVSHVTYTYHVTDIIPVMLIGVIGGILGSLYNHLLHKVLRLYNLINEKGKIHKVLLSLTVSLFTSVCLYGLPFLAKCKPCDPSIDEICPTNGRSGNFKQFHCPKGYYNDLATLLLTTNDDAVRNLFSSNTPNEFGMGSLWIFFVLYCILGLFTFGIATPSGLFLPIILMGAAYGRMLGAAMGSYTSIDQGLYAVLGAAALMAGSMRMTVSLCVIFLELTNNLLLLPITMIVLLIAKTVGDSFNPSIYDIILHLKGLPFLEANPEPWMRNLTVGELGDAKPPVVTLQGVEKVSNIVDVLKNTTHNAFPVLDEAEVPQVGLATGATELHGLILRAHLVKVLKKRWFLTEKRRTEEWEVREKFPWDELAEREDNFDDVAITSAEMEMYVDLHPLTNTTPYTVMENMSVAKALVLFRQVGLRHLLIVPKIQASGMCPVVGILTRQDLRAYNILQAFPLLEKSKGGKTH

SEQ ID NO: 3 - DNA, 232 bp, Carnation etched ring virus
agcttgcatgcctgcaggtcgagcttttaggattccatagtgataagatatgttcttatctaaacaaaaaagcagcgtcggcaaaccatacagctgtccacaaaaaggaaaggctgtaataacaagcggacccagcttctcagtggaagatactttatcagacactgaataatggatggaccctaccacgattaaagaggagcgtctgtctaaagtaaagtagagcgtcttt

SEQ ID NO: 4 - DNA, 22 bp, Arabidopsis thaliana
atggtggaagaagatttaaacc

SEQ ID NO: 5 - DNA, 19 bp, Arabidopsis thaliana
tcaatgtgtctttccacct

SEQ ID NO: 6 - DNA, 18 bp, Escherichia coli
tgtaaaacgacggccagt

SEQ ID NO: 7 - DNA, 19 bp, Escherichia coli
aggaaacagctatgaccat

(19) United States
(12) Patent Application Publication
(10) Pub. No.: US 2015/0075547 A1
(43) Pub. Date: Mar. 19, 2015

(54) TRANSGENIC PLANTS HAVING LOWER NITRATE CONTENT IN LEAVES

(71) Applicant: BRITISH AMERICAN TOBACCO (INVESTMENTS) LIMITED, London (GB)

(72) Inventors: Pascaline LE LAY, London (GB); Juan-Pablo SANCHEZ TAMBURRINO, London (GB); Susan DAVENPORT, London (GB)

(21) Appl. No.: 14/386,709

(22) PCT Filed: Mar. 19, 2013
(86) PCT No.: PCT/GB2013/050707

§ 371 Date: Sep. 19, 2014

(30) Foreign Application Priority Data
Mar. 20, 2012 (GB) 1204862.5

(51) Int. Cl. C12N 15/82; A24B 13/00; A24D 1/00

(52) U.S. Cl. CPC C12N 15/8243; C12N 15/8251; A24D 1/00; A24B 13/00
USPC 131/352; 435/320.1; 800/278; 800/298; 435/419; 800/306; 800/317; 800/317.3

(57) ABSTRACT

The present invention relates to genetic constructs, which can be used in the preparation of transgenic plants. The constructs can have the ability of reducing nitrate concentration in the plant, in particular the plant's leaves, and for inducing a senescence-like phenotype. The invention extends to plant cells transformed with such constructs, and to the transgenic plants themselves. The invention also relates to methods of producing transgenic plants, and to methods of reducing nitrate content in plants. The invention also relates to harvested plant leaves, for example tobacco leaves, that have been transformed with the genetic constructs, and to various tobacco articles, such as smoking articles, comprising such harvested plant leaves.

### TECHNICAL FIELD

The present invention relates to genetic constructs, which can be used in the preparation of transgenic plants. The constructs can have the ability of reducing nitrate concentration in the plant, in particular the plant's leaves. The invention extends to plant cells transformed with such constructs, and to the transgenic plants themselves. The invention also relates to methods of producing transgenic plants, and to methods of reducing nitrate content in plants. The invention also provides methods for modifying plant amino acid profiles. The invention also relates to harvested plant leaves, for example tobacco leaves, that have been transformed with the genetic constructs, and to various tobacco articles, such as smoking articles, comprising such harvested plant leaves.

### BACKGROUND

Nitrogen assimilation is of fundamental importance to the growth of plants. Of all the mineral nutrients required by plants, nitrogen is required in the greatest abundance. The main forms of nitrogen taken up by plants in the field are nitrate and ammonia, the principle components of nitrogenous fertilizers. Plants take up either nitrate or ammonium ions from the soil, depending on availability. Nitrate will be more abundant in well-oxygenated, non-acidic soils, whilst ammonium will predominate in acidic or water-logged soils. Experiments on growth parameters of tobacco clearly demonstrated that relative growth rate, chlorophyll content, leaf area and root area increased dramatically in response to increasing nitrate supply.

Plants have developed an efficient nitrogen uptake system in order to cope with the large variation in nitrate content of cultivated soils. Plant roots take up nitrate and ammonia by the action of specific nitrate transporters (NTR), which are divided into two gene families, the NRT1 gene family and the NRT2 gene family. Both gene families coexist in plants, and they are believed to act cooperatively to take up nitrate from the soil to assist with distributing it to cells found throughout the plant. However, once inside the cell, very little is known about the mechanisms that are employed for the transport of nitrate to different cellular compartments.

After entry into the cell, it is believed that nitrate accumulates in vacuoles leading to concentrations as high as 50 mM, which is 25 times higher than the concentration of nitrate found within the cytoplasm. Vacuolar nitrate contributes to the maintenance of homeostasis in cytosolic nitrate. AtCLC-a, an anion/proton exchanger, which belongs to the Arabidopsis CLC protein family has been shown to play a role in vacuolar nitrate transport. AtCLC-a is a known nitrate-proton exchanger, and is responsible for loading nitrate into vacuoles. Knock-out of AtCLC-a in Arabidopsis causes a 50% reduction in its nitrate accumulation capacity compared to wild-type plants. This indicates that there are additional genes which may also be responsible for loading nitrate into plant vacuoles.

Seven homologues of the CLC protein family have been identified within Arabidopsis, and they are referred to as AtCLC-a to AtCLC-g. Based on sequence identity, AtCLC-a, -b, -c, -d and -g each define a separate phylogenetic branch with the highest homology with the subfamily of mammalian CLCs. The AtCLCs are ubiquitously expressed in plants. However, the functional role of these proteins is far from being understood.

In addition to AtCLC-a, AtCLC-c is also believed to be a major component of nitrate accumulation pathway in plants. The shoots of Arabidopsis plants, which contain a transposon insertion for AtCLC-c, possess a lower nitrate concentration compared to the roots of wild-type plants. However, unlike AtCLC-a mutants, the roots of AtCLC-c mutants also posses an altered chloride concentration compared to wild-type plants, which suggests that AtCLC-c exhibits less anion specificity than AtCLC-a. Furthermore, AtCLC-d, which is expressed in the trans-Golgi network of plant cells and co-localizes with a V-type ATPase, is believed to play a role in the development of plant roots. This is supported by the finding that T-DNA insertion of a non-functional AtCLC-d mutant in Arabidopsis plants impairs root growth, with little or no effect on chloride ion content, nitrate content or cellular morphology.

Once in the cell, the nitrate is reduced in the cytosol by the cytoplasmic enzyme nitrate reductase (NR) to nitrite. Newly formed nitrite is then transported into the chloroplast and rapidly reduced to ammonium by nitrite reductase (NiR). Ammonium then enters the glutamine synthetase/glutamate synthase cycle (GS/GOGAT), where it is incorporated into the amino acid pool. The mechanism by which nitrite is transported from the cytosol to the chloroplast is not known. It has been postulated that passive diffusion may account for its entry into chloroplasts, and that this may partly be due to the presence of transporter proteins that are expressed on the surface of chloroplasts, such as: (i) CsNitr1 (Cucumis sativus nitrite transporter)—a nitrate transporter protein that has been identified on the inner membrane of cucumber chloroplast envelopes; (ii) At1g68570—an orthologue of CsNitr1. Over-expression of a non-functional form of the At1g68570 polypeptide in Arabidopsis causes excessive accumulation of nitrite in transgenic plants compared to wild-type plants; and (iii) AtCLC-e—an Arabidopsis CLC protein family member that is expressed on the surface of thylakoid membranes within chloroplasts. Knock-out of clc-e from Arabidopsis plants results in a reduction of nitrate accumulation as well as an increase in the accumulation of nitrite within transgenic Arabidopsis plants compared to wild-type plant cells.

However, the relatively low concentration of nitrite within the cytosol of Arabidopsis makes passive diffusion unlikely to be the mechanism that is responsible for the entry of nitrite into chloroplasts. In addition, it is difficult to conclude whether AtCLC-e actually plays a role in regulating intracellular nitrate fluxes as knock-out of AtCLC-e from Arabidopsis also influences the expression of several other genes that are also implicated in the regulation of intracellular nitrate levels.

The regulation of the activities of nitrate transporters, and nitrate and nitrite reductases is critical in controlling primary nitrogen assimilation throughout the plant, and has a significant impact on the growth and development of the plant. High levels of nitrate accumulate during periods of low temperature and/or solar irradiation (for example, in greenhouse crops during the winter), when there is less photosynthetic capacity to assimilate the stored nitrate, or as a result of high nitrate levels in the soil. An increase in nitrate levels can have a number of deleterious consequences, not only in terms of plant growth, but also in terms of human or animal health where the plant is consumed, as well as environmental consequences. Many of the adverse consequences of nitrate accumulation are mediated through the production of nitrite.

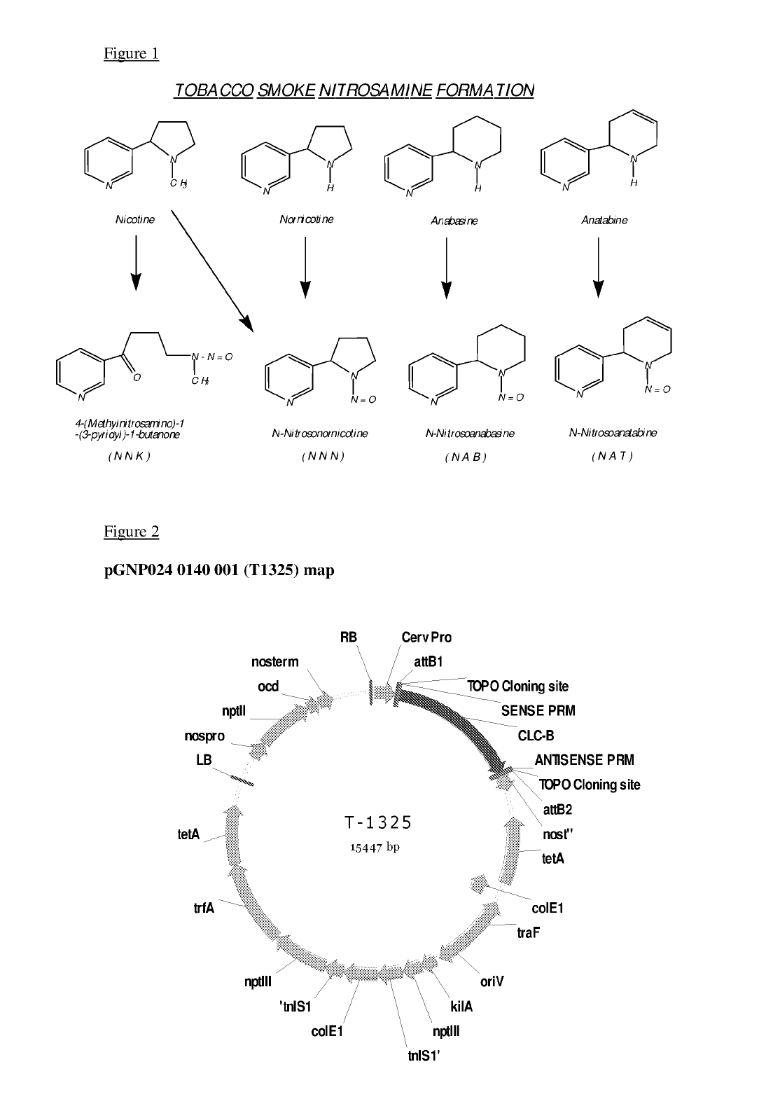

Therefore, to prevent excessive nitrate accumulation, one strategy would be to decrease nitrate storage in plants. This could be performed by modifying the storage of nitrate within the vacuoles of plants, and would be useful in the tobacco industry. It is well known that residual nitrogen in tobacco leaves contributes to the formation of nitrosamines, as illustrated in FIG. 1. In particular, nitrate and nitrite act as precursors to tobacco-specific nitrosamine (TSNA) formation in cured leaf.

In the tobacco industry, the processing of the tobacco leaves involves the removal of petioles and midribs of the cured leaves that are believed to act as nitrate storage organs, which are devoid of flavour and high in TSNAs.

Also, the formation of nitrosamines in the stomach is a result of endogenous nitrosation. Oral bacteria chemically reduce nitrate consumed in food and drink to nitrite, which can form nitrosating agents in the acidic environment of the stomach. These react with amines to produce nitrosamines and cause DNA strand breaks or cross linking of DNA. Another problem associated with an excess of nitrate is the formation of methaemoglobin which gives rise to blue baby syndrome, where the oxygen carrying capacity of haemoglobin is blocked by nitrite, causing chemical asphyxiation in infants.

As a consequence of these health concerns, a number of regulatory authorities have set limits on the amount of nitrate allowed in leafy green vegetables such as spinach and lettuce (e.g. European Commission Regulation 653/2003), depending on the time of harvest. These limits have resulted in any produce with a high nitrate content being unmarketable. Consequently, there have been efforts to reduce nitrate content of plants by managing the application of nitrogen-containing fertilisers or improved systems of crop husbandry. Some authorities have also set limits on the amounts of nitrate in drinking water.

There is therefore a need for means for alleviating the adverse effects associated with nitrate accumulation in plants. With this in mind, the inventors have developed a series of genetic constructs, which may be used in the preparation of transgenic plants, which exhibit surprisingly reduced nitrate concentrations.

### SUMMARY OF THE INVENTION

Thus, according to a first aspect of the invention, there is provided a genetic construct comprising a promoter operably linked to a coding sequence encoding a polypeptide, which is an anion/proton exchanger having nitrate transporter activity, with the proviso that the promoter is not a cauliflower mosaic virus 35S promoter.

As described in the Examples, the inventors have investigated the remobilisation of nitrogen in a plant, with a view to developing plants which exhibit decreased concentrations of nitrate, especially in the leaves. The inventors prepared a number of genetic constructs (see FIG. 2), in which a gene encoding an anion/proton exchanger protein having nitrate transporter activity was placed under the control of a promoter, which was not the CAMV 35S promoter. The promoter may however be a constitutive promoter or a tissue-specific promoter.

In one embodiment, the coding sequence in the construct may encode the Arabidopsis anion/proton exchanger, CLC-b. The cDNA sequence encoding one embodiment of the Arabidopsis CLC-b anion/proton exchanger is provided herein as SEQ ID No.1, as follows:

[SEQ ID No. 1]
ATGGTGGAAGAAGATTTAAACCAGATTGGTGGTAATAGTAATTACAATGGAGAAGGAGGCGACCCAGA
GAGCAACACACTTAACCAACCTCTAGTTAAGGCTAATCGAACACTTTCTTCAACTCCACTTGCTTTGG
TTGGTGCCAAAGTTTCCCATATCGAAAGCTTGGACTATGAAATAAACGAGAACGATCTGTTTAAGCAT
GATTGGAGAAAAAGATCAAAGGCACAAGTACTTCAATACGTGTTCTTGAAATGGACGTTAGCTTGTCT
TGTTGGTCTTTTCACTGGTTTAATCGCTACTCTCATCAACTTAGCTGTTGAAAACATCGCCGGCTATA
AGCTTTTAGCCGTTGGTCACTTCCTCACTCAAGAAAGATATGTTACAGGTCTGATGGTGCTTGTTGGG
GCGAATTTGGGACTGACGTTGGTGGCGTCTGTGCTTTGTGTGTGCTTTGCTCCTACGGCGGCTGGACC
TGGAATCCCTGAGATCAAAGCTTATCTTAATGGTGTAGATACTCCCAACATGTTTGGTGCTACTACTA
TGATCGTTAAGATTGTTGGAAGCATTGGAGCGGTTGCAGCTGGACTTGATCTAGGTAAAGAGGGTCCT
CTAGTTCACATTGGAAGCTGCATAGCTTCTTTGCTTGGACAAGGTGGAACAGACAACCACCGTATCAA
GTGGCGGTGGCTTCGTTACTTCAACAACGATAGAGACCGCAGGGATCTGATTACATGTGGCTCAGCTG
CAGGAGTGTGTGCAGCCTTCAGGTCACCTGTTGGAGGTGTACTTTTCGCCCTCGAGGAAGTTGCTACT
TGGTGGAGAAGTGCCTTATTGTGGCGGACTTTCTTCAGCACAGCGGTTGTTGTGGTTGTTCTAAGAGA
GTTCATAGAGATCTGCAATTCAGGGAAGTGTGGGTTGTTTGGAAAAGGAGGGCTAATCATGTTTGATG
TGAGTCATGTAACTTATACTTACCATGTAACTGATATAATCCCTGTCATGTTGATTGGTGTAATCGGT
GGAATTCTTGGGAGCCTGTACAATCATCTTCTGCATAAAGTTCTCAGGCTTTACAATCTCATCAATGA
GAAGGGTAAGATCCATAAGGTGCTTCTCAGTCTTACAGTATCACTCTTTACATCTGTTTGCCTTTATG
GCCTTCCTTTCTTAGCGAAATGCAAGCCTTGTGACCCCTCGATAGATGAGATATGCCCGACGAATGGA
AGATCGGGTAACTTCAAACAGTTCCATTGCCCTAAAGGTTACTACAATGATCTAGCTACTCTGCTTCT
CACCACCAACGATGATGCTGTCAGAAACCTTTTCTCTTCCAACACTCCCAATGAGTTTGGTATGGGTT
CCCTTTGGATATTCTTTGTGCTATACTGCATCTTGGGGCTTTTCACATTTGGTATTGCAACACCGTCT
GGTCTCTTCCTCCCCATCATCCTCATGGGTGCTGCATATGGCCGAATGCTTGGCGCTGCAATGGGATC
ATACACAAGTATTGACCAAGGGCTTTATGCTGTCCTTGGTGCAGCTGCACTCATGGCTGGATCGATGA
GAATGACTGTGTCACTCTGTGTTATATTCCTTGAACTCACCAACAACCTTCTTTTGCTTCCTATAACG
ATGATCGTGCTTCTGATAGCCAAAACTGTGGGAGACAGCTTTAACCCGAGTATATATGACATCATCTT
GCATCTAAAGGGCTTACCTTTCTTAGAAGCAAATCCAGAGCCGTGGATGAGGAACCTCACCGTTGGTG
AGCTTGGTGATGCTAAGCCCCCGGTTGTAACCCTGCAAGGTGTTGAAAAGGTTTCAAATATAGTTGAT
GTGCTAAAGAACACGACGCATAATGCATTCCCTGTTTTAGATGAAGCAGAAGTACCTCAAGTGGGTCT
AGCAACTGGGGCTACAGAACTCCACGGGTTGATCTTGAGAGCGCACCTCGTTAAAGTTCTGAAAAAGA
GATGGTTCTTGACAGAGAAAAGAAGAACAGAGGAGTGGGAGGTCAGAGAAAAGTTTCCATGGGATGAA
TTGGCTGAAAGAGAAGACAACTTTGACGACGTGGCCATCACAAGCGCTGAAATGGAAATGTATGTCGA
TCTTCATCCTCTCACCAACACAACACCTTACACAGTCATGGAGAACATGTCAGTGGCCAAGGCTTTAG
TACTTTTCCGGCAAGTGGGACTCCGGCATTTGCTTATTGTTCCCAAGATTCAAGCCTCAGGAATGTGT
CCTGTGGTAGGGATCTTAACCAGACAGGACCTAAGGGCATACAACATTCTACAAGCCTTTCCTCTCTT
GGAAAAATCCAAAGGTGGAAAGACACATTGA

The polypeptide sequence of the Arabidopsis CLC-b anion/proton exchanger is provided herein as SEQ ID No.2, as follows:

[SEQ ID No. 2]
MVEEDLNQIGGNSNYNGEGGDPESNTLNQPLVKANRTLSSTPLALVGAKVSHIESLDYEIN
ENDLFKHDWRKRSKAQVLQYVFLKWTLACLVGLFTGLIATLINLAVENIAGYKLLAVGHFL
TQERYVTGLMVLVGANLGLTLVASVLCVCFAPTAAGPGIPEIKAYLNGVDTPNMFGATTMI
VKIVGSIGAVAAGLDLGKEGPLVHIGSCIASLLGQGGTDNHRIKWRWLRYFNNDRDRRDLI
TCGSAAGVCAAFRSPVGGVLFALEEVATWWRSALLWRTFFSTAVVVVVLREFIEICNSGKC
GLFGKGGLIMFDVSHVTYTYHVTDIIPVMLIGVIGGILGSLYNHLLHKVLRLYNLINEKGK
IHKVLLSLTVSLFTSVCLYGLPFLAKCKPCDPSIDEICPTNGRSGNFKQFHCPKGYYNDLA
TLLLTTNDDAVRNLFSSNTPNEFGMGSLWIFFVLYCILGLFTFGIATPSGLFLPIILMGAA
YGRMLGAAMGSYTSIDQGLYAVLGAAALMAGSMRMTVSLCVIFLELTNNLLLLPITMIVLL
IAKTVGDSFNPSIYDIILHLKGLPFLEANPEPWMRNLTVGELGDAKPPVVTLQGVEKVSNI
VDVLKNTTHNAFPVLDEAEVPQVGLATGATELHGLILRAHLVKVLKKRWFLTEKRRTEEWE
VREKFPWDELAEREDNFDDVAITSAEMEMYVDLHPLTNTTPYTVMENMSVAKALVLFRQVG
LRHLLIVPKIQASGMCPVVGILTRQDLRAYNILQAFPLLEKSKGGKTH*

The * in the above sequence refers to the stop codon at the 3′ end of the sequence, and is required for termination of expression. The polypeptide may comprise an amino acid sequence as set out in SEQ ID No.2, or a functional variant or fragment or orthologue thereof. Accordingly, the coding sequence, which encodes the polypeptide, which is an anion/proton exchanger having nitrate transporter activity, may comprise a nucleic acid sequence substantially as set out in SEQ ID No.1, or a functional variant or fragment or orthologue thereof.

The promoter may be capable of inducing RNA polymerase to bind to, and start transcribing, the coding sequence encoding the polypeptide having nitrate transporter activity. The promoter in constructs of the invention may be a constitutive, non-constitutive, tissue-specific, developmentally-regulated or inducible/repressible promoter.

A constitutive promoter directs the expression of a gene throughout the various parts of the plant continuously during plant development, although the gene may not be expressed at the same level in all cell types. Examples of known constitutive promoters include those associated with the rice actin 1 gene (Zhang et al., 1991, Plant Cell, 3, 1155-65) and the maize ubiquitin 1 gene (Cornejo et al., 1993, Plant Molec. Biol., 23, 567-581). Constitutive promoters such as the Carnation Etched Ring Virus (CERV) promoter (Hull et al., 1986, EMBO J., 5, 3083-3090) are particularly preferred in the present invention.

A tissue-specific promoter is one which directs the expression of a gene in one (or a few) parts of a plant, usually throughout the life-time of those plant parts. The category of tissue-specific promoter commonly also includes promoters whose specificity is not absolute, i.e. they may also direct expression at a lower level in tissues other than the preferred tissue. Examples of tissue-specific promoters known in the art include those associated with the patatin gene expressed in potato tuber, and the high molecular weight glutenin gene expressed in wheat, barley or maize endosperm.

A developmentally-regulated promoter directs a change in the expression of a gene in one or more parts of a plant at a specific time during plant development, e.g. during senescence. The gene may be expressed in that plant part at other times at a different (usually lower) level, and may also be expressed in other plant parts.

An inducible promoter is capable of directing the expression of a gene in response to an inducer. In the absence of the inducer the gene will not be expressed. The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. A developmentally-regulated promoter can be described as a specific type of inducible promoter responding to an endogenous inducer produced by the plant or to an environmental stimulus at a particular point in the life cycle of the plant. Examples of known inducible promoters include those associated with wound response, temperature response, and chemically induced.

The promoter may be obtained from different sources including animals, plants, fungi, bacteria, and viruses, and different promoters may work with different efficiencies in different tissues. Promoters may also be constructed synthetically. Therefore, examples of suitable promoters include the Carnation Etch Ring Virus (CERV) promoter, the pea plastocyanin promoter, the rubisco promoter, the nopaline synthase promoter, the chlorophyll a/b binding promoter, the high molecular weight glutenin promoter, the α,β-gliadin promoter, the hordein promoter, the patatin promoter, or a senescence-specific promoter. For example, a suitable senescence-specific promoter may be one which is derived from a senescence-associated gene (SAG), and may be selected from a group consisting of SAG12, SAG13, SAG101, SAG21 and SAG18.

Preferably, the promoter is the CERV promoter, as shown in the construct illustrated in FIG. 2. The Carnation Etch Ring Virus (CERV) promoter will be known to the skilled technician, (Hull et al., EMBO J., 5, 3083-3090). The DNA sequence encoding the CERV promoter is 232 bp long, and is referred to herein as SEQ ID No.3, as follows:

[SEQ ID No. 3]
AGCTTGCATGCCTGCAGGTCGAGCTTTTAGGATTCCATAGTGATAAGATATGTTCTTATCTA
AACAAAAAAGCAGCGTCGGCAAACCATACAGCTGTCCACAAAAAGGAAAGGCTGTAATAACA
AGCGGACCCAGCTTCTCAGTGGAAGATACTTTATCAGACACTGAATAATGGATGGACCCTAC
CACGATTAAAGAGGAGCGTCTGTCTAAAGTAAAGTAGAGCGTCTTT

Therefore, the promoter in the construct of the invention may comprise a nucleotide sequence substantially as set out in SEQ ID No.3, or a functional variant or functional fragment thereof. The CERV promoter may be obtained from Cauliovirus or a plant species such as Dianthus caryophyllus (i.e. carnation) showing signs of the cauliovirus. In embodiments where the promoter is the CERV promoter, it will be appreciated that the promoter may comprise each of the bases 1-232 of SEQ ID No.3. However, functional variants or functional fragments of the promoter may also be used in genetic constructs of the invention.

A “functional variant or functional fragment of a promoter” can be a derivative or a portion of the promoter that is functionally sufficient to initiate expression of any coding region that is operably linked thereto. For example, in embodiments where the promoter is based on the CERV promoter, the skilled technician will appreciate that SEQ ID No.3 may be modified, or that only portions of the CERV promoter may be required, such that it would still initiate gene expression in the construct.

Functional variants and functional fragments of the promoter may be readily identified by assessing whether or not transcriptase will bind to a putative promoter region, and then lead to the transcription of the coding region into the polypeptide having nitrate transporter activity. Alternatively, such functional variants and fragments may be examined by conducting mutagenesis on the promoter, when associated with a coding region, and assessing whether or not gene expression may occur.

The coding sequence, which encodes the polypeptide which is an anion/proton exchanger having nitrate transporter activity, may be derived from any suitable source, such as a plant. The coding sequence may be derived from a suitable plant source, for example from Arabidopsis spp., Oryza spp., Populus spp. or Nicotiana spp. The coding sequence may be derived from Arabidopsis thaliana, Oryza sativa, Populus tremula or Nicotiana tabacum. It will be appreciated that orthologues are genes or proteins in different species that evolved from a common ancestral gene by speciation, and which retain the same function.

The inventors have created a construct in which the CERV promoter has been used to drive expression of the Arabidopsis thaliana anion/proton exchanger protein, CLC-b.

The construct may be capable of decreasing, in a plant transformed with a construct of the invention, the concentration of nitrate by at least 5%, 10%, 15%, 18%, 20%, 32%, 35%, 38%, 40%, 50%, 60% or 63% compared to the concentration of nitrate in the wild-type plant (i.e. which has not been transformed with a construct of the invention), preferably grown under the same conditions.

The construct may be capable of decreasing, in a plant transformed with the construct, the concentration of 4-(Methylnitrosamino)-1-(3-pyridyl)-1-butanone (NNK) by at least %, 20%, 30%, 40%, 50%, 60%, 61%, 62%, 65%, 69%, 71% or 75% compared to the concentration of NNK in the wild-type plant, preferably grown under the same conditions.

The construct may be capable of decreasing, in a plant transformed with the construct, the concentration of N-Nitrosonornicotine (NNN) by at least 10%, 20%, 30%, 40%, 50%, 60%, 70%, 71%, 75%, 78%, 80%, 82%, 84%, 85%, 88%, 90% or 94% compared to the concentration of NNN in the wild-type plant, preferably grown under the same conditions.

The construct may be capable of decreasing, in a plant transformed with the construct, the concentration of N-Nitrosoanatabine (NAT) by at least 5%, 6%, 10%, 20%, 23%, 24%, 30%, 40%, 46%, 45%, 48%, 50%, 60%, 70%, 80% or 85% compared to the concentration of NAT in the wild-type plant, preferably grown under the same conditions.

The construct may be capable of decreasing, in a plant transformed with the construct, the concentration of total tobacco-specific nitrosamines (TSNA) by at least 10%, 20%, 30%, 40%, 50%, 56%, 60%, 64%, 65%, 70% or 75% compared to the concentration of total TSNA in the wild-type plant, preferably grown under the same conditions.

Preferably, the construct is capable of decreasing the concentration of any of the compounds selected from a group of compounds including nitrate, NNK, NNN, NAT and total TSNA, in a leaf or stem from a plant of a T0, T1 and/or T2 plant population, preferably grown under the same conditions.

The construct may be capable of decreasing the concentrations of any of these compounds (i.e. nitrate, amino acids involved in nitrogen assimilation, total TSNA, NNN, NAT or NNK) in a leaf located at a lower, middle or upper position on the plant. “Lower position” can mean in the lower third of the plant (for example leaf number 4 or from the base of the plant), “upper position” can mean in the upper third of the plant (for example leaf number 14 or 15 from the base of the plant), and “middle position” can mean the central third of the plant between the lower and upper positions (for example leaf number 10 or 11 from the base of the plant). At the time of sampling, the total number of leaves is approximately 20.

Genetic constructs of the invention may be in the form of an expression cassette, which may be suitable for expression of the coding sequence encoding an anion/proton exchanger in a host cell. The genetic construct of the invention may be introduced into a host cell without it being incorporated in a vector. For instance, the genetic construct, which may be a nucleic acid molecule, may be incorporated within a liposome or a virus particle. Alternatively, a purified nucleic acid molecule (e.g. histone-free DNA or naked DNA) may be inserted directly into a host cell by suitable means, e.g. direct endocytotic uptake. The genetic construct may be introduced directly into cells of a host subject (e.g. a plant) by transfection, infection, microinjection, cell fusion, protoplast fusion or ballistic bombardment. Alternatively, genetic constructs of the invention may be introduced directly into a host cell using a particle gun. Alternatively, the genetic construct may be harboured within a recombinant vector, for expression in a suitable host cell.

Hence, in a second aspect, there is provided a recombinant vector comprising the genetic construct according to the first aspect.

The recombinant vector may be a plasmid, cosmid or phage. Such recombinant vectors are highly useful for transforming host cells with the genetic construct of the invention, and for replicating the expression cassette therein. The skilled technician will appreciate that genetic constructs of the invention may be combined with many types of backbone vector for expression purposes. The backbone vector may be a binary vector, for example one which can replicate in both E. coli and Agrobacterium tumefaciens. For example, a suitable vector may be a pBIN plasmid, such as pBIN19 (Bevan M., 1984, Nucleic Acids Research 12:8711-21).

Recombinant vectors may include a variety of other functional elements in addition to the promoter (e.g. a CERV), and the coding sequence encoding an anion/proton exchanger with nitrate transporter activity. For instance, the recombinant vector may be designed such that it autonomously replicates in the cytosol of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged.

The recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. The vector may also comprise DNA involved with regulating expression of the coding sequence, or for targeting the expressed polypeptide to a certain part of the host cell, e.g. the chloroplast. Hence, the vector of the second aspect may comprise at least one additional element selected from a group consisting of: a selectable marker gene (e.g. an antibiotic resistance gene); a polypeptide termination signal; and a protein targeting sequence (e.g. a chloroplast transit peptide).

Examples of suitable marker genes include antibiotic resistance genes such as those conferring resistance to Kanamycin, Geneticin (G418) and Hygromycin (npt-II, hyg-B); herbicide resistance genes, such as those conferring resistance to phosphinothricin and sulphonamide based herbicides (bar and suI respectively; EP-A-242246, EP-A-0249637); and screenable markers such as beta-glucuronidase (GB2197653), luciferase and green fluorescent protein (GFP). The marker gene may be controlled by a second promoter, which allows expression in cells, which may or may not be in the seed, thereby allowing the selection of cells or tissue containing the marker at any stage of development of the plant. Suitable second promoters are the promoter of nopaline synthase gene of Agrobacterium and the promoter derived from the gene which encodes the 35S cauliflower mosaic virus (CaMV) transcript. However, any other suitable second promoter may be used.

The various embodiments of genetic constructs of the invention may be prepared using the cloning procedure described in the Examples, which may be summarised as follows. The cDNA version of the genes encoding the anion/proton exchanger may be amplified from cDNA templates by PCR using suitable primers, for example SEQ ID No's 4 and 5. PCR products may then be examined using agarose gel electrophoresis. The PCR products may then be ligated into a suitable vector for cloning purposes, for example that which is available under the trade name TOPO pCR8 from Invitrogen. Vectors harbouring the PCR products may be grown up in a suitable host, such as E. coli. E. coli colonies and inserts in plasmids showing the correct restriction enzyme digest pattern may be sequenced using suitable primers.

E. coli colonies carrying pCR8-TOPO-cDNA for Atclc-b may be cultured to produce a suitable amount of each plasmid, which may then be purified. The plasmids may then be digested to release a DNA fragment encoding the Atclc-b gene, which may then be cloned into a vector, such as a pBNP plasmid (van Engelen et al., 1995, Transgenic Research, 4:288-290), harbouring a suitable promoter, for example the CERV promoter.

The resultant Atclc-b construct, contained the CERV promoter and was named CRVAtCLC-b. Embodiments of the vector according to the second aspect may be substantially as set out in FIG. 2. The inventors believe that they are the first to have developed a method for decreasing nitrate concentrations in plant leaves using the expression of the exogenous anion/proton exchanger gene Atclc-b in a transgenic plant.

Hence, in a third aspect, there is provided a method of decreasing the nitrate concentration in the leaves of a test plant to below that of the corresponding nitrate concentration in leaves of a wild-type plant cultured under the same conditions, the method comprising:—

(i) transforming a plant cell with the genetic construct according to the first aspect, or the vector according to the second aspect; and

(ii) regenerating a plant from the transformed cell.

In a fourth aspect of the invention, there is provided a method of producing a transgenic plant which transports nitrate out of a leaf at a higher rate than a corresponding wild-type plant cultured under the same conditions, the method comprising:—

(i) transforming a plant cell with the genetic construct according to the first aspect, or the vector according to the second aspect; and

(ii) regenerating a plant from the transformed cell.

In a fifth aspect, there is provided a method for producing a transgenic plant, the method comprising introducing, into an unmodified plant, an exogenous gene encoding a polypeptide, which is an anion/proton exchanger having nitrate transporter, wherein expression of the anion/proton exchanger encoded by the exogenous gene reduces the nitrate concentration in the leaves of the transgenic plant relative to the concentration of nitrate in leaves of the unmodified plant.

The position of a leaf in relation to the rest of the plant (i.e. whether it is regarded as being within the “lower” position, the “top” position or the “middle” position) is important for tobacco growers. The physiology, and therefore, the quality and the flavour of a leaf are strongly related to its position within a plant. As a plant approaches flowering, a process called remobilization occurs, and it involves the transport of nutrients, such as amino acids and nitrogenous compounds, from the base of the plant towards the top of the plant. Remobilized nutrients will be used as an energy source for seed production. Consequently, the lower leaves will have a different nitrogen content compared to the upper leaves of the plant, which is illustrated by a different amino acid profile. Lower leaves are called “source leaves” and the top leaves are called “sink leaves”. The middle leaves are fully expanded mature green leaves.

With respect to some plants, such as tobacco, by removing the flower head of the plant, changes in leaf nutrient metabolism can be generated. These changes allow the remobilized nutrients to be used in the leaves, and result in thickened leaves, general growth of the leaves and the production of nitrogen-rich secondary metabolites, many of which are the precursors of the flavours that are later found in cured leaves. Therefore, constructs of the invention may be used to modify the flavour of a transgenic plant.

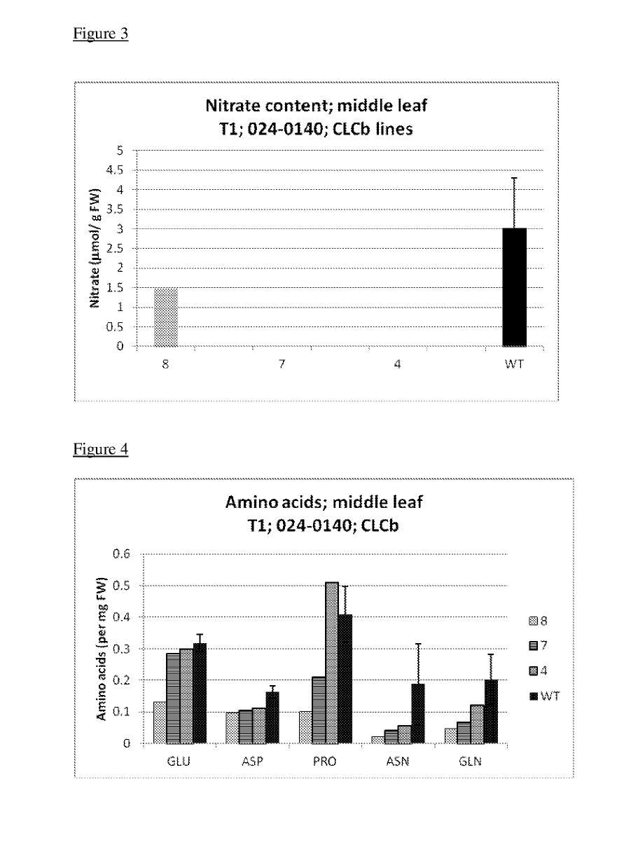

As shown in FIG. 3, the inventors were surprised to observe that the genetic constructs according to the invention may also be capable of modulating (i.e. increasing and/or decreasing) the concentration of certain amino acids that are known to involved in nitrate metabolism (e.g. Gln, Asn, Asp, Glu and/or Pro), in the leaves of a transgenic plant, which are found in the upper, middle or lower position, compared to corresponding leaves that are found in a wild-type plant grown under the same conditions.

Accordingly, in a sixth aspect, there is provided a method of modulating the profile of amino acids involved in the nitrogen assimilation of leaves of a test plant compared to the amino acid profile of corresponding leaves of a wild-type plant cultured under the same conditions, the method comprising:—

(i) transforming a plant cell with the genetic construct according to the first or second aspect, or the vector according to the third aspect; and

(ii) regenerating a plant from the transformed cell.

In an seventh aspect, there is provided a method of modulating the profile of amino acids involved in the nitrogen assimilation pathway of a harvested leaf taken from a transgenic plant, compared to the amino acid profile of a corresponding harvested leaf taken from a wild-type plant cultured under the same conditions, wherein the leaf is harvested from a transgenic plant produced by the method according to either the fourth or fifth aspect.

According to the invention, amino acids involved in the nitrogen assimilation pathway of plants and their leaves may comprise glutamine (Gln), asparagine (Asn), aspartic acid (Asp), glutamic acid (Glu) or proline (Pro), and so any of the profile or any or all of these amino acids may be modulated.

The construct may be capable of decreasing or increasing, in a plant transformed with the construct, the concentration of at least one amino acid involved in the nitrogen assimilation pathway by at least 10%, 20%, 30%, 40%, 50%, 56%, 60%, 64%, 65%, 70% or 75% compared to the concentration of the at least one amino acid in a wild-type plant grown under the same conditions.

Preferably, the construct results in the decrease in concentration of the amino acid. Preferably, the construct may be capable of decreasing the concentration of the amino acids, Glu, Asp, Pro, Gln and/or Asn, in the leaves (preferably the middle leaves) of a transgenic plant compared to corresponding leaves that are found in a wild-type plant grown under the same conditions.

In a eighth aspect, there is provided a transgenic plant comprising the genetic construct according to the first aspect, or the vector according to the second aspect.

In an ninth aspect, there is provided a transgenic plant comprising an exogenous gene encoding a polypeptide, which is an anion/proton exchanger having nitrate transporter activity, wherein the nitrate concentration in leaves of the transgenic plant is reduced compared to the nitrate concentration in leaves of an unmodified plant.

In an tenth aspect, there is provided use of an exogenous nucleic acid sequence encoding a polypeptide, which is an anion/proton exchanger having nitrate transporter activity, for reducing nitrate concentration in plant leaves by transformation of the plant with the exogenous nucleic acid sequence.

The term “unmodified plant” can mean a plant before transformation with an exogenous gene or a construct of the invention. The unmodified plant may therefore be a wild-type plant.

The term “exogenous gene” can mean the gene that is transformed into the unmodified plant is from an external source, i.e. from a different species to the one being transformed. The exogenous gene may have a nucleic acid sequence substantially the same or different to an endogenous gene encoding an anion/proton exchanger in the unmodified plant. The exogenous gene may be derived from cDNA sequence encoding Atclc-b gene or an orthologue thereof. The exogenous gene may form a chimeric gene, which may itself constitute a genetic construct according to the first aspect. The exogenous gene may encode an anion/proton exchanger having the amino acid sequence substantially as set out in SEQ ID No.2, or a functional variant or fragment or orthologue thereof. The exogenous gene may comprise the nucleotide sequence substantially as set out in SEQ ID No.1, or a functional variant or fragment or orthologue thereof.

The methods and uses of the invention may comprise transforming a test plant cell or unmodified plant cell with a genetic construct according to the first aspect, a vector according to the second aspect, or the exogenous gene described herein.

Thus, in a eleventh aspect, there is provided a host cell comprising the genetic construct according to the first aspect, or the recombinant vector according to the second aspect.

The cell may be a plant cell. The cell may be transformed with a genetic construct, vector or exogenous gene according to the invention, using known techniques. Suitable means for introducing the genetic construct into the host cell may include use of a disarmed Ti-plasmid vector carried by Agrobacterium by procedures known in the art, for example as described in EP-A-0116718 and EP-A-0270822. A further method may be to transform a plant protoplast, which involves first removing the cell wall and introducing the nucleic acid, and then reforming the cell wall. The transformed cell may then be grown into a plant.

Preferably, and advantageously, the methods and uses according to the invention do not compromise the health or fitness of the test or transgenic plant that is generated.

The transgenic or test plants according to invention may include the Brassicaceae family, such as Brassica spp. The plant may be Brassica napus (oilseed rape). Further examples of transgenic or test plants include the family Poales, such as Triticeae spp. The plant may be Triticum spp. (wheat). Increasing the grain protein content in wheat may result in increased volume of food products comprising wheat, such as bread.

Further examples of suitable transgenic or test plants according to the invention may include the Solanaceae family of plants which include, for example jimson weed, eggplant, mandrake, deadly nightshade (belladonna), capsicum (paprika, chili pepper), potato and tobacco. One example of a suitable genus of Solanaceae is Nicotiana. A suitable species of Nicotiana may be referred to as tobacco plant, or simply tobacco.

Further examples of suitable transgenic or test plants according to the invention may include leafy crops such as the Asteraceae family of plants which, for example, include lettuce (Lactuca sativa). Another example may include the Chenopodiaceae family of plants, which includes Spinacia oleracea and Beta vulgaris, i.e. spinach and chards, respectively.

Tobacco may be transformed with constructs, vectors and exogenous genes of the invention as follows.

Nicotiana tabacum is transformed using the method of leaf disk co-cultivation essentially as described by Horsch et al. (Science 227: 1229-1231, 1985).

The youngest two expanded leaves may be taken from 7-week old tobacco plants and may be surface-sterilised in 8% Domestos™ for 10 minutes and washed (3 rinses) times with sterile distilled water. Leaf disks may be cut using a number 6 cork borer and placed in the Agrobacterium suspension, containing the appropriate binary vectors (according to the invention), for approximately two minutes. The discs may be gently blotted between two sheets of sterile filter paper. Ten disks may be placed on MS 3% sucrose+2.2 μM BAP+0.27 μM NAA plates, which may then be incubated for 2 days in the growth room. Discs may be transferred to plates of MS+3% sucrose+2.2 μM BAP+0.27 μM NAA supplemented with 500 g/l Cefotaxime and 100 g/l kanamycin. The discs may be transferred onto fresh plates of above medium after 2 weeks. After a further two weeks, the leaf disks may be transferred onto plates containing LS+3% sucrose+0.5 μM BAP supplemented with 500 mg/l Cefotaxime and 100 mg/l kanamycin. The leaf disks may be transferred onto fresh medium every two weeks. As shoots appear, they may be excised and transferred to jars of LS+3% sucrose+0.5 μM BAP supplemented with 500 mg/l claforan. The shoots in jars may be transferred to LS+3% sucrose+250 mg/l Cefotaxime after approximately 3 weeks. After a further 3-4 weeks the plants may be transferred to LS+3% sucrose (no antibiotics) and rooted. Once the plants are rooted they may be transferred to soil in the greenhouse.

In a twelfth aspect, there is provided a plant propagation product obtainable from the transgenic plant according to either the sixth or ninth aspect.

A “plant propagation product” may be any plant matter taken from a plant from which further plants may be produced. Suitably, the plant propagation product may be a seed. The plant propagation product may preferably comprise a construct or vector according to the invention or an exogenous gene.

In an thirteenth aspect of the invention, there is provided a harvested leaf containing a lower level of nitrate than the corresponding level of nitrate in a harvested leaf taken from a wild-type plant cultured under the same conditions, wherein the leaf is harvested from the transgenic plant according to either the sixth or ninth aspect, or produced by the method according to either the fourth or fifth aspect.

In a fourteenth aspect of the invention, there is provided a tobacco product comprising nitrate-reduced tobacco obtained from a mutant tobacco plant comprising the construct of the first aspect or the vector of the second aspect, which mutant is capable of decreasing the concentration of nitrate in its leaves.

It is preferred that the mutant tobacco plant from which the tobacco in the tobacco product is derived comprises a construct, vector or exogenous gene according to the invention.

The tobacco product may be smokeless tobacco product, such as snuff. The tobacco product may be an oral tobacco product deliverable by the mouth. The tobacco product may be moist, and may be snus. However, the tobacco product may also be a smoking article.

Thus, in a fifteenth aspect, there is provided a smoking article comprising nitrate-reduced tobacco obtained from a mutant tobacco plant comprising the construct of the first aspect or the vector of the second aspect, which mutant is capable of decreasing the concentration of nitrate in its leaves.

Nitrate-reduced tobacco can include tobacco in which the nitrate concentration is less than the corresponding concentration in a wild-type plant cultured under the same conditions. Such a smoking article may comprise tobacco obtained from a mutant tobacco plant, which may have been transformed with a genetic construct according to the first aspect of the invention, or a vector according to the second aspect, or an exogenous gene. Preferably, the mutant tobacco plant comprises the anion-proton exchanger AtCLC-b, which may comprise the amino acid sequence substantially as set out in SEQ ID No.2, or a functional variant or fragment or orthologue thereof. ATCLC-b may comprise the nucleotide sequence substantially as set out in SEQ ID No.1, or a functional variant or fragment or orthologue thereof.

The term “smoking article” can include smokable products, such as rolling tobacco, cigarettes, cigars and cigarillos whether based on tobacco, tobacco derivatives, expanded tobacco, reconstituted tobacco or tobacco substitutes and also heat-not-burn products.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/polynucleotide/polypeptide sequence”, “functional variant” and “functional fragment”, can be a sequence that has at least 40% sequence identity with the amino acid/polynucleotide/polypeptide sequences of any one of the sequences referred to herein, for example 40% identity with the gene identified as SEQ ID No.1 (which encodes one embodiment of an anion/proton exchanger), or 40% identity with the polypeptide identified as SEQ ID No.2 (i.e. one embodiment of an anion/proton exchanger).

Amino acid/polynucleotide/polypeptide sequences with a sequence identity which is greater than 65%, more preferably greater than 70%, even more preferably greater than 75%, and still more preferably greater than 80% sequence identity to any of the sequences referred to is also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences is then calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No.1, or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/cm % SDS at approximately 20-65° C. Alternatively, a substantially similar polypeptide may differ by at least 1, but less than 5, 10, 20, 50 or 100 amino acids from the sequences shown in SEQ ID No.2.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

In order to address various issues and advance the art, the entirety of this disclosure shows by way of illustration various embodiments in which the claimed invention(s) may be practiced and provide for superior method for reducing the nitrate concentration in the leaves of transgenic plants. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows the chemical structures of various tobacco smoke nitrosamines, 4-(Methylnitrosamino)-1-(3-pyridyl)-1-butanone (NNK), N-Nitrosonornicotine (NNN), N-Nitrosoanabasine (NAB) and N-Nitrosoanatabine (NAT);

FIG. 2 is a plasmid map of one embodiment of a construct according to the invention, known as pGNP024 0140 001. The construct includes the Atclc-b anion/proton exchanger gene under the control of the Carnation Etched Ring Virus (CERV) promoter;

FIG. 3 shows the amino acid profile in the middle leaf of three T1 lines (i.e. 4, 7 and 8) harbouring the promoter CERV::CLCb construct (Wild type [WT] Virginia40 acted as control); and

FIG. 4 shows the concentration of nitrate in the middle leaves of three T1 lines (i.e. 4, 7 and 8) harbouring the promoter CERV::CLCb construct (Wild type [WT] Virginia40 acted as control).

### DETAILED DESCRIPTION & EXAMPLES

The inventors have developed a construct, as shown in FIG. 2, and have used it to create transgenic plant lines which over-express the anion/proton exchanger gene Arabidopsis thaliana clc-b under the control of the constitutive promoter, Carnation Etched Ring Virus (CERV) promoter (Hull et al., 1986, EMBO J., 5, 3083-3090).

### Example 1

### Isolation of Arabidopsis thaliana Anion/Proton Exchanger Gene

The Arabidopsis thaliana anion/proton exchange gene used in these experiments was Atclc-b.

Design of Primers

The full length genomic sequence coding for the Arabidopsis thaliana anion/proton exchanger CLC-b was identified (Accession Number for the sequences was: AAD29679. Primers for use in PCR to isolate the genomic sequence were designed, which were tailed at the 5′ end with a 4 bp spacer and suitable restriction sites. attB restriction sites were generated at the 5′ and 3′ end of the fragment to enable the cloning of the fragments into appropriate vectors.

It will be appreciated by the skilled person that other PCR primers could be designed incorporating the required features of the primers and alternative restriction enzyme sites.

Isolation of Arabidopsis cDNA Encoding CLC-b

Arabidopsis thaliana var. Columbia RNA was extracted from the rosette leaves of 3-week old plants using the Qiagen RNA Easy kit. Briefly, RNA was extracted from leaf samples using a QIAGEN RNA easy kit (QIAGEN Ltd., Crawley, UK), following the manufacturer's instructions. This method provided large amounts of very clean RNA suitable for gene isolation and cloning strategies. cDNA was prepared from the RNA samples using Retroscript first strand synthesis kit (Ambion) following manufacturer's instructions using random primers.

Isolation of Dc-b Anion/Proton Exchanger DNA Fragments

The sequence of Arabidopsis clc-b is 2355 bp long (accession number ADD29679). cDNA encoding Arabidopsis clc-b was amplified with primer pairs SEQ ID NO.4 and SEQ ID NO.5, which generated attB restriction sites at the 5′ end and attB restriction sites at the 3′ end of the fragment.

Forward
[SEQ ID NO. 4]
ATGGTGGAAGAAGATTTAAACC
Reverse
[SEQ ID NO. 5]
TCAATGTGTCTTTCCACCT

PCR Conditions for Arabidopsis clc-b

Cycle program: 1 cycle of 94° C. for 5 minutes, followed by 30 cycles of 94° C. for 30 seconds, 60° C. for 30 seconds and 72° C. for 2 minutes, this was followed by 1 cycle of 72° C. for 5 minutes, followed by hold at 4° C. The band was isolated using Advantage 2 polymerase (Clonetech) following manufacturer's instructions. Gel purification of the fragments was carried out by running the fragments on a 1% Tris Acetate EDTA (TAE) agarose crystal violet gel using a SWAT UV free kit (Invitrogen).

An aliquot of the PCR reactions were then analysed by agarose gel electrophoresis. Reactions were precipitated and then stored. Clc-b anion/exchanger DNA fragments were then cloned into pCR8 TOPO vector (available from Invitrogen), as described below.

Ligation Reactions for Arabidopsis clc-b

1 μl pCR8 TOPO was taken with 1 μl salt solution, and 4 μl PCR reaction. The mixture was left at room temperature for 10 mins. 4 μl of the ligation reaction mixture was taken with TOP10 E. coli cells, and then left on ice for 5 mins. The cells were heat-shocked at 42° C., and then left on ice for 5 min. The cells were then incubated in 250 μl SOC for 2 hours. The cells were then plated onto agar plates containing spectinomycin (100 μg/ml) and left overnight at 37° C. Cells containing plasmids grew into colonies. 10 single colonies were picked and cultured in LB medium and observed for each gene sequence. Mini preps (Qiagen) were made for each individual colony and a restriction digest using EcoRI and XhoI was used to determine if the gene had been incorporated into the pCR8-TOPO vector.

Individual colonies were picked for each sequence containing the expected sized PCR fragment. Individual colonies were then grown up and plasmid DNA was extracted for sequence analysis. These were sent to Beckman Coulter for sequencing with the primers shown below (i.e. SEQ ID No:6 and SEQ ID No:7).

MI3F (Forward)
[SEQ ID NO. 6]
TGT AAA ACG ACG GCC AGT
M13R (Reverse)
[SEQ ID NO. 7]
AGG AAA CAG CTA TGA CCA T

Sequence Analysis

Analysis of the sequence showed that the clones contained the anion/exchanger gene Atclc-b.

### Example 2

### Construction of Vectors for Tobacco Transformation

Cloning of cDNA Encoding Atclc-b into a Binary Vector

pCR8 plasmids containing the clc-b gene were recombined with the pGBNPCERV Gateway destination vector (Invitrogen) together with LR clonase II enzyme mix and TE buffer. This was incubated at 25° C. overnight and then 1 μl of proteinase K was added to stop the reaction. The transformed vector was subsequently used to transform E. coli electrocompetent cells. The pBNP vector is an in-house vector created from the pBNP binary vector (van Engelen et al., 1995, Transgenic Research, 4:288-290) that was made Gateway ready using the Gateway conversion kit (Invitrogen), containing the CERV promoter and the nopaline synthase terminator. Cells containing the plasmid were selected on kanamycin plates. Clones were then isolated and the DNA was extracted and analysed by restriction digestion followed by sequencing.

The CERV promoter is a constitutive promoter of the caulimovirus group of plant viruses. It was isolated and characterised in 1986 by Hull et al. and is characteristic of CaMV (Hull et al., 1986), but has little sequence similarity with the CaMV 35S promoter.

The following binary vector was produced: pGNP024 0140 001 (T1325) (see FIG. 2): Carnation Etch Ring Virus (CERV) promoter: Clc-b cDNA: Nos terminator. The binary vector was then transformed into Agrobacterium tumefaciens LBA 4404 by electroporation. This was performed by mixing 40 μl of A. tumefaciens electrocompetent cells and 0.5 μg of plasmid DNA, and placing in a pre-cooled cuvette. The cells were then electroporated at 1.5 Volts, boo Ohms and 25 μFD. 1 ml of 2YT media was added to the cuvette and the mixture was decanted into a 30 ml universal container and incubated at 28° C. for 2 hours in a shaking incubator. 100 μl of cells were then plated onto kanamycin (50 μg/ml) and streptomycin (100 μg/ml) LB agar plates. The plates were left to incubate for 2 days at 28° C.

### Example 3

### Transformation of Tobacco

Burley PH2517 plants were transformed with pGNP024 0140 001 using the method of leaf disk co-cultivation, as described by Horsch et al. (Science 227: 1229-1231, 1985). The youngest two expanded leaves were taken from 7-week old tobacco plants and were surface-sterilised in 8% Domestos for 10 minutes and washed 3 times with sterile distilled water. Leaf disks were then cut using a number 6 cork borer and placed in the transformed Agrobacterium suspension for approximately two minutes. The discs were then gently blotted between two sheets of sterile filter paper. 10 disks were placed on MS 3% sucrose+2.2 μM BAP+0.27 μM NAA plates, which were then incubated for 2 days in the growth room. Discs were then transferred to plates of MS+3% sucrose+2.2 μM BAP+0.27 μM NAA supplemented with 500 g/l Cefotaxime and 100 g/l kanamycin.

The discs were transferred onto fresh plates of the above medium after 2 weeks. After a further two weeks the leaf disks were transferred onto plates containing LS+3% sucrose+0.5 μM BAP supplemented with 500 mg/l Cefotaxime and 100 mg/l kanamycin. The leaf disks were transferred onto fresh medium every two weeks. As shoots appeared, they were excised and transferred to jars of LS+3% sucrose+0.5 μM BAP supplemented with 500 mg/l Cefotaxime. The shoots in jars were transferred to LS+3% sucrose+250 mg/l Cefotaxime after approximately 3 weeks. After a further 3-4 weeks, the plants were finally transferred to LS+3% sucrose (no antibiotics) and rooted. Once the plants were rooted they were transferred to soil in the greenhouse.

### Example 4

### Analysis of Tobacco “Middle” Leaf Nitrate Content (T1 Plants)

Quantification of nitrate and/or nitrite levels in wild-type and transgenic Virginia40 plants was performed using HPLC. This method for determining nitrate concentrations in plant tissues is described in Sharma et al., 2008 (Malaria Journal, 7: pp 71). HPLC provides highly accurate measurements of nitrate and/or nitrite levels from plant samples and also reduces the concerns associated with handling hazardous agents due to the increased level of automation associated with the methodology.

Materials are:

Running Buffer: 5 mM K₂HPO₄, 25 mM KH₂PO₄ at pH3

Extraction Buffer: 5 mM K₂HPO₄, 25 mM KH₂PO₄ at pH3

Method: Firstly, 2 ml of the phosphate buffer is added to 250-300 mg of ground leaf material and homogenised in mortar with a pestle. These ratios can be modified according to the expected level of nitrate. The homogenate is then centrifuged at 16000 rpm at +4° C. for 10 minutes. 1 ml of the supernatant is then filtered through a syringe filter (0.2 μm) into an HPLC vial. Nitrate and Nitrite standard curves were constructed with concentrations range of 0-1 mM for nitrate and 0 to 100 μM for nitrite. The injection volume is 20 μl.

The peak identification is done according to peak timing. Peak timing is variable depending on column age and a number of other factors. Thus standards should be used to assess peak position and related this to peak run off time in samples.

The nitrate results illustrated in FIG. 3 show that there is a lowering of leaf nitrate concentration in the transformed plants harbouring the CRV-AtClcb construct of the invention. Although they do not wish to be bound by theory, the inventors hypothesise that the AtClcb protein is acting as a nitrogen remobiliser. This results in the leaves being depleted of nitrate.

### Example 5

### Analysis of Tobacco “Middle” Leaf Amino Acid Content (T1 Plants)

The physiology of a leaf is dependent on its position in relation to the rest of the plant. Therefore, tobacco growers must bear this information in mind when considering what flavour a leaf may possess.

During flowering, a process called remobilization occurs, which results in the transport of nutrients, such as amino acids and nitrogenous compounds, out from the base of the plant towards the top of the plant. In addition, remobilized nutrients will be used as an energy source for seed production. Therefore, lower and upper leaves will have a different nitrogen content illustrated by a different amino acid profile.

Amino acids are routinely analysed using the EZ: Faast LC/MS kit supplied by Phenomenex. The kit provides reagent, consumable to allow simultaneous derivatization of amino acids from a sample of tissue such that they may be separated and detected within a single run of the QTrap LC/MS.

The principles of the method are:

The procedure consists of a solid phase extraction step followed by a derivatization and a liquid/liquid extraction; derivatized samples are then analysed by liquid chromatography-mass spectrometry. The solid phase extraction is performed via a sorbent packed tip that binds amino acids while allowing interfering compounds to flow through. Amino acids on sorbent are then extruded into the sample vial and quickly derivatized with reagent at room temperature in aqueous solution. Derivatized amino acids concomitantly migrate to the organic layer for additional separation from interfering compounds. Organic layer is then removed, evaporated and re-dissolved in aqueous mobile phase and analyzed on a LC/MS system.

All the reagents and supplies (including the HPLC column) are components of the kit. All the steps of the procedure are detailed on the User's manual KH0-7337 and KH0-7338 which are used as a protocol.

FIG. 4 summarises the effect of AtClcb over-expression on the concentration of Glu, Asp, Pro, Gln and Asn (i.e. amino acids believed to be involved in the nitrogen assimilation pathway of plants) in three plant lines (i.e. 4, 7 and 8). This figure clearly shows that plants harbouring the AtClcb an anion/proton exchanger construct exhibit a significant reduction (compared to the middle leaves of their wild-type counterpart) in the concentration of all the amino acids measured.

In view of the reduced nitrate content of the test plant leaves, as shown in Example 4, and the reduced amino acid content of the middle leaves of the three plant lines analysed, the inventors conclude that there is reduced nitrate available for TSNA formation in the leaves of plants that over-express CRV-AtClcb, which would clearly be advantageous for tobacco plants. Furthermore, manipulation of the amino acid profiles can be used to modify the flavour of tobacco.

1. A genetic construct comprising a promoter operably linked to a coding sequence encoding a polypeptide, which is an anion/proton exchanger having nitrate transporter activity, wherein
a) the polypeptide comprises an amino acid sequence substantially as set out in SEQ ID No.2, or a functional variant or fragment or orthologue thereof, or
b) the coding sequence comprises a nucleic acid sequence substantially as set out in SEQ ID No.1, or a functional variant or fragment or orthologue thereof,
and wherein the promoter is selected from the one of the group consisting of: a Carnation Etch Ring Virus (CERV) promoter, a pea plastocyanin promoter, a rubisco promoter, a nopaline synthase promoter, a chlorophyll a/b binding promoter, a high molecular weight glutenin promoter, an α,β-gliadin promoter, a hordein promoter, a patatin promoter, and a senescence-specific promoter.

2. The genetic construct according to claim 1, wherein the promoter is a Carnation Etch Ring Virus (CERV) promoter, optionally wherein the promoter comprises a nucleotide sequence substantially as set out in SEQ ID No.3, or a functional variant or functional fragment thereof.

3. The genetic construct according to claim 1, wherein the coding sequence is derived from Arabidopsis spp., Oryza spp., Populus spp. or Nicotiana spp.

4. The genetic construct according to claim 1, wherein the coding sequence is derived from Arabidopsis thaliana, Oryza sativa, Poputus tremula or Nicotiana tabacum.

5. A recombinant vector comprising the genetic construct according to claim 1.

6. A method of decreasing nitrate concentration in leaves of a test plant to below that of the corresponding nitrate concentration, in leaves of a wild-type plant cultured under the same conditions, the method comprising:
(i) transforming plant cell of the test plant with the genetic construct according to the construct according to claim 1; and
(ii) regenerating a plant from the transformed cell, wherein the regenerated transformed plant has decreased nitrate concentration in its leaves when compared to the wild-type plant.

7. A method of producing a transgenic plant which transports nitrate out of a leaf at a higher rate than a corresponding wild-type plant cultured under the same conditions, the method comprising:
(1) transforming a plant cell of the test plant with the genetic construct according to the construct according to claim 1, and
(ii) regenerating a plant from the transformed cell, wherein the regenerated transformed plant has decreased nitrate concentration in its leaves when compared to the wild-type plant.

8. A method for producing a transgenic plant, the method comprising introducing, into an unmodified plant, an exogenous gene encoding a polypeptide, which is an anion/proton exchanger having nitrate transporter activity, wherein
a) the polypeptide comprises an amino acid sequence substantially as set out in SEQ ID No.2, or a functional variant or fragment or orthologue thereof, or
b) the exogenous gene comprises the nucleotide sequence substantially as set out in SEQ ID No.1, or a functional variant or fragment or orthologue thereof,
and wherein expression of the nitrate transporter encoded by the exogenous gene reduces nitrate concentration in leaves of the transgenic plant relative to the concentration of nitrate in leaves of the unmodified plant.

9. A transgenic plant comprising the genetic construct according to claim 1.

10. A transgenic plant comprising an exogenous gene encoding a polypeptide, which is an anion/proton exchanger having nitrate transporter activity, wherein
a) the polypeptide comprises an amino acid sequence substantially as set out in SEQ ID No.2, or a functional variant or fragment or orthologue thereof, or
b) the exogenous gene comprises the nucleotide sequence substantially as set out in SEQ ID No.1, or a functional variant or fragment or orthologue thereof,
and wherein nitrate concentration in leaves of the transgenic plant is reduced compared to nitrate concentration in leaves of an unmodified plant.

11. (canceled)

12. A host cell comprising the genetic construct according to claim 1.

13. The host cell according to claim 12, wherein the cell is a plant cell.

14. The method according to claim 6, wherein the plant is selected from the group consisting of Brassicaceae family, Pales family, Solanaceae family, Nicotiana genus, Asteraceae family, and Chenopodiaceae family.

15. The method according to claim 7, wherein the plant is selected from the group consisting of Brassicaceae family, Pales family, Solanaceae family, Nicotiana genus, Asteraceae family, and Chenopodiaceae family.

16. The method according to claim 8, wherein the plant is selected from the group consisting of Brassicaceae family, Pales family, Solanaceae family, Nicotiana genus, Asteraceae family, and Chenopodiaceae family.

17. The transgenic plant according to claim 9, wherein the plant is selected from the group consisting of Brassicaceae family, Pales family, Solanaceae family, Nicotiana genus, Asteraceae family, and Chenopodiaceae family.

18. The transgenic plant according to claim 10, wherein the plant is selected from the group consisting of Brassicaceae family, Pales family, Solanaceae family, Nicotiana genus, Asteraceae family, or Chenopodiaceae family.

19. A plant propagation product obtainable from the transgenic plant according to claim 9.

20. A harvested leaf containing a lower level of nitrate than the corresponding level of nitrate in a harvested leaf taken from a wild-type plant cultured under the same conditions, wherein the leaf is harvested from the transgenic plant according to either claim 9.

21. A tobacco product comprising nitrate-reduced tobacco obtained from a mutant tobacco plant comprising the construct according to claim 1, which mutant is capable of decreasing the concentration of nitrate in its leaves.

22. The tobacco product according to claim 21, wherein the tobacco product is a smokeless tobacco product, or an oral tobacco product deliverable by the mouth, or a smoking article.

23. A smoking article comprising nitrate-reduced tobacco obtained from a mutant tobacco plant comprising the construct according to claim 1, which mutant is capable of decreasing the concentration of nitrate in its leaves.

24. A method of modulating a profile of amino acids involved in nitrogen assimilation of leaves of a test plant compared to the amino acid profile of corresponding leaves of a wild-type plant cultured under the same conditions, the method comprising:
(i) transforming a plant cell of the test plant with the genetic construct according to claim 1; and
(ii) regenerating a plant from the transformed cell, wherein the profile of amino acids involved in the nitrogen assimilation of leaves of the regenerated transformed plant is modulated compared to the amino acid profile of corresponding leaves of the wild-type plant.

25. A method of modulating a profile of amino acids involved in nitrogen assimilation pathway of a harvested leaf taken from a transgenic plant, compared to the amino acid profile of a corresponding harvested leaf taken from a wild-type plant cultured under the same conditions, wherein the leaf is harvested from a transgenic plant produced by the method according to claim 7.

26. The method according to claim 24, wherein the amino acids involved in the nitrogen assimilation pathway of plants and their leaves may comprise glutamine (Gin), asparagine (Asn), aspartic acid (Asp), glutamic acid (Glu) or proline (Pro).

27. The method according to claim 24, wherein the construct is capable of decreasing or increasing, in a plant transformed with the construct, the concentration of at least one amino acid involved in the nitrogen assimilation pathway by at least 10%, 20%, 30%, 40%, 50%, 56%, 60%, 64%, 65%, 70% or 75% compared to the concentration of the at least one amino acid in a wild-type plant grown under the same conditions.

28. The method according to claim 24, wherein the construct is capable of decreasing the concentration of the amino acids, Glu, Asp, Pro, Gin and/or Asn, in the middle leaves of a transgenic plant compared to corresponding leaves that are found in a wild-type plant grown under the same conditions.
